(19) United States
(12) Patent Application Publication
(10) Pub. No.: US 2024/0118280 A1
(43) Pub. Date: Apr. 11, 2024

(54) METHODS AND COMPOSITIONS FOR PROTEIN FINGERPRINTING

(71) Applicant: Quantum-Si Incorporated, Branford, CT (US)

(72) Inventors: Omer Ad, Madison, CT (US); Robert E. Boer, Westbrook, CT (US)

(73) Assignee: Quantum-Si Incorporated, Branford, CT (US)

(21) Appl. No.: 18/335,963

(22) Filed: Jun. 15, 2023

Related U.S. Application Data
(60) Provisional application No. 63/352,422, filed on Jun. 15, 2022.

(51) Int. Cl. G01N 33/573; G01N 33/543

(52) U.S. Cl. CPC G01N 33/573; G01N 33/54366; G01N 2333/912; G01N 2333/96436

(57) ABSTRACT

Aspects of the disclosure relate to methods and compositions for identifying, isolating, and/or sequencing a target protein or peptide from a sample of proteins or peptides.

### CROSS-REFERENCE TO RELATED APPLICATION

This application claims priority under 35 U.S.C. § 119(e) to U.S. Provisional Patent Application No. 63/352,422, filed on Jun. 15, 2022, which is hereby incorporated by reference in its entirety.

### REFERENCE TO AN ELECTRONIC SEQUENCE LISTING

The contents of the electronic sequence listing (R070870128US01-SEQ-MSB.xml; Size: 2,326 bytes; and Date of Creation: Jun. 15, 2023) is herein incorporated by reference in its entirety.

### TECHNICAL FIELD

The present disclosure relates to methods and compositions for identifying, isolating, and sequencing a target protein or peptide from a plurality of proteins or peptides.

### BACKGROUND

Proteomics has emerged as important in the study of biological systems. These analyses of an individual organism or sample type can provide insights into cellular processes and response patterns, which lead to improved diagnostic and therapeutic strategies. The complexity surrounding protein compositions and modification present challenges in determining large-scale sequencing information for a biological sample. Improved and more convenient techniques and systems for identifying, isolating, and sequencing a subset of proteins or peptides from complex protein compositions are desirable.

### SUMMARY

Aspects of this disclosure relate to methods and compositions for the identification and/or detection of one or more target molecules in a sample. In some embodiments, a target molecule is a peptide, a protein, or a fragment or derivative thereof. Through the use of the methods and/or the compositions of the instant disclosure, target molecules may, in some embodiments, be more readily identified and/or detected in a sample. The subject matter of the present invention involves, in some cases, interrelated products, alternative solutions to a particular problem, and/or a plurality of different uses of one or more systems and/or articles.

Aspects of the disclosure relate to a method of identifying a target protein, the method comprising: contacting a sample comprising the target protein with a chemical probe that comprises a functional unit that specifically interacts with the target protein; loading the sample on a chip comprising a plurality of sample wells; detecting the sample on the chip, wherein at least a subset of the plurality of sample wells comprises the target protein bound to the chemical probe; and sequencing the contents of at least the subset of the plurality of sample wells comprising the target protein bound to the chemical probe, thereby identifying the target protein.

In some embodiments, the sample is a biological sample. In some embodiments, the biological sample is derived from blood, a tissue sample, a bacterial sample, or a sample of the microbiome. In some embodiments, the sample is a whole proteome.

Aspects of the disclosure relate to a method of protein sequencing, the method comprising: immobilizing a target protein bound to a chemical probe at a base of a well within a chip comprising a plurality of wells, optionally the chemical probe comprises a functional unit that specifically interacts with the target protein; detecting the target protein bound to the chemical probe in at least a subset of the plurality of wells; sequencing the contents of at least the subset of the plurality of wells comprising the target protein bound to the chemical probe, thereby identifying the target protein. In some embodiments, the target protein bound to the chemical probe is immobilized to the base of the well via a secondary complex. In some embodiments, the secondary complex is a streptavidin-biotin complex. In some embodiments, the chemical probe is a small molecule. In some embodiments, the small molecule is a drug (e.g., a drug that interacts with the active site of an enzyme), a synthetic analogue of alkyl-CoAs, an NTP (e.g., ATP, CTP, GTP, TTP), an NDP (e.g., ADP, CDP, GDP, TDP), an NMP (e.g., AMP, CMP, GMP, TMP), a nucleotide, a small molecule that binds covalently to the protein (e.g., at an active site), a small molecule that binds non-covalently to the protein (e.g., at an allosteric site). In some embodiments, the functional unit comprises a chemical warhead, optionally the chemical warhead is a an electrophilic warhead, a photocaged radical warhead, or nucleophilic warhead. In some embodiments, the chemical probe further comprises an orthogonal reactive group, optionally the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

In some embodiments, the method is automated or manual. In some embodiments, the automated method occurs in a single instrument. In some embodiments, the target protein is identified at a concentration lower than a concentration required by conventional LC-MS technologies.

Aspects of the disclosure relate to a chip comprising a plurality of wells and a target protein bound to a chemical probe immobilized to a base of at least a subset of the plurality of wells, optionally the chemical probe comprises a functional unit that specifically interacts with the target protein. In some embodiments, the plurality of wells is a number of wells selected from the group consisting of: 96 wells, 384 wells, 1,536 wells, or more wells. In some embodiments, the target protein is a peptide derived from a sample comprising a plurality of peptides. In some embodiments, the chemical probe is a small molecule. In some embodiments, the small molecule is a drug (e.g., a drug that interacts with the active site of an enzyme), a synthetic analogue of alkyl-CoAs, an NTP (e.g., ATP, CTP, GTP, TTP), an NDP (e.g., ADP, CDP, GDP, TDP), an NMP (e.g., AMP, CMP, GMP, TMP), a nucleotide, a small molecule that binds covalently to the protein (e.g., at an active site), a small molecule that binds non-covalently to the protein (e.g., at an allosteric site). In some embodiments, the target protein bound to a chemical probe is immobilized to the base of the well via a secondary complex. In some embodiments, the secondary complex is a streptavidin-biotin complex.

In some embodiments, the functional unit comprises a chemical warhead, optionally the chemical warhead is an electrophilic warhead, a photocaged radical warhead, or nucleophilic warhead. In some embodiments, the chemical probe further comprises an orthogonal reactive group, optionally the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

Aspects of the disclosure relate to a method of identifying two or more protein homologues, the method comprising: contacting a sample comprising the two or more protein homologues with a chemical probe that comprises a functional unit that binds to a shared feature of the two or more protein homologues; loading the sample on a chip comprising a plurality of sample wells; detecting the sample on the chip, wherein at least a subset of the plurality of samples wells comprises at least one of the two or more protein homologues; and sequencing the contents of at least the subset of the plurality of sample wells thereby identifying each protein of the plurality of target proteins.

In some embodiments, the two or more protein homologues comprise amino acid sequences that share at least 80%, 85%, 90%, or 95% sequence similarity. In some embodiments, the shared feature of the two or more protein homologues is a protein domain, active site, allosteric site, or post-translational modification.

In some embodiments, the chemical probe is a small molecule. In some embodiments, the small molecule is a drug (e.g., a drug that interacts with the active site of an enzyme), a synthetic analogue of alkyl-CoAs, an NTP (e.g., ATP, CTP, GTP, TTP), an NDP (e.g., ADP, CDP, GDP, TDP), an NMP (e.g., AMP, CMP, GMP, TMP), a nucleotide, a small molecule that binds covalently to the protein (e.g., at an active site), a small molecule that binds non-covalently to the protein (e.g., at an allosteric site). In some embodiments, the functional unit comprises a chemical warhead, optionally the chemical warhead is a an electrophilic warhead, a photocaged radical warhead, or nucleophilic warhead. In some embodiments, the chemical probe further comprises an orthogonal reactive group, optionally the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

Aspects of the disclosure relate to a composition comprising a peptide attached to a nucleic acid via a linker, wherein a first region of the linker is covalently attached or bound to the peptide using a functional group that specifically interacts with the peptide, and wherein a second region of the linker is covalently attached or bound to the nucleic acid by an orthogonal reactive group. In some embodiments, the linker further comprises a secondary complex. In some embodiments, the secondary complex is a streptavidin-biotin complex. In some embodiments, the composition is immobilized on a surface (e.g., the surface of a chip well). In some embodiments, the functional unit comprises a chemical warhead, optionally the chemical warhead is a an electrophilic warhead, a photocaged radical warhead, or nucleophilic warhead.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

Non-limiting embodiments of the present invention are described by way of example with reference to the accompanying figures, which are schematic and are not intended to be drawn to scale unless otherwise indicated. In some embodiments of the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows an example schematic for protein fingerprinting. A desired druggable unit (e.g., whole cell proteome, plasma, serum, etc.) is, in some embodiments, exposed to a chemical probe comprising a functional drug molecule (“warhead”) bearing a synthetic handle (“CLICK handle”, e.g., a functional unit). The protein is then, in some embodiments, processed by cysteine reduction and capping and proteolytic digestion. The synthetic handle is then, in some embodiments, used to produce peptides bound to streptavidin-DNA for immobilization on a chip. The chip may be then loaded and a photobleaching step applied. All apertures showing a bleach-step contain the drug molecule covalently bound to the druggable unit. Finally, the druggable unit may be sequenced and a target protein identified.

FIG. 2A shows Ibrutinib-yne comprising an electrophilic reactive group (“warhead”) and an alkyne moiety (“CLICK handle”) for high-throughput identification.

FIG. 2B shows Ibrutinib-yne for high-throughput identification bound to EGFR kinase via a cysteine bond.

FIGS. 3A-3B shows a PAGE analysis of a sample comprising EGFR kinase incubated with an Ibrutinib-yne structure and a control sample comprising EGFR kinase alone. FIG. 3A shows a PAGE experiment stained with Coomassie blue to identify all protein in both samples.

FIG. 3B shows a PAGE experiment stained with AF647-azide to demonstrate that only the protein bearing Ibrutinib-yne reacted with AF647-azide.

FIG. 4 shows a schematic for protein fingerprinting using an EGFR kinase domain. The EGFR kinase domain is incubated with Ibrutinib-yne or a DMSO control at 37° C. for 1 hour. The EGFR kinase domain bound to Ibrutinib-yne is exposed to cysteine reduction and capping using TCEP and chloroacetamide at 37° C. for 1 hour. The product is desalted with a 7 kDa zeba column and exposed to proteolytic digestion by trypsin at 37° C. for 16 hours. The product is further conjugated with bis-biotin-DNA-SS-azide at room temperature for 30 minutes and finally exposed to DTT at 65° C. for 10 minutes.

FIGS. 5A-5C show LC-MS plots of DMSO control (FIG. 5A), Ibrutinib-yne (FIG. 5B), and Ibrutinib-yne with a DNA control molecule and DTT (FIG. 5C). The blue box represents unmodified peptide, the green box represents peptide bound to Ibrutinib-yne, and the red box represents peptide-Ibrutinib-yne-DNA adduct following disulfide reduction.

FIGS. 6A-6B show the structures of a chemical probe comprising a fluorophosphonate azide (FP Azide) before (FIG. 6A) and after (FIG. 6B) a reaction with Granzyme B (a serine protease).

FIGS. 6C-6D shows a PAGE analysis of a sample comprising Granzyme B incubated with FP Azide and a control sample comprising Granzyme B alone. FIG. 6C shows a PAGE experiment stained with Coomassie blue to identify all protein in both samples. FIG. 6D shows a PAGE experiment stained with Cy3-DBCO to demonstrate that only the protein bearing FP Azide reacted with Cy3-DBCO.

FIG. 7 shows a cutaway perspective view of an example chip.

### DETAILED DESCRIPTION

In some aspects, the disclosure provides methods and compositions for identifying, detecting, and/or sequencing a target molecule or target molecules within a sample. In some embodiments, a target molecule is a peptide, a protein, or a fragment or derivative thereof. Some such embodiments may accelerate analysis of target peptide samples from within a sample of a plurality of peptides or proteins. In some embodiments, methods and compositions described herein facilitate the incubation, digestion, functionalization (e.g. via derivatization), quenching (e.g., via contact with functionalized solid substrates), and/or purification of peptide samples. These embodiments may provide advantages for the preparation and analysis of peptide samples. In some embodiments, peptide samples comprise proteins or peptides, and analysis of the peptide samples may permit sequencing of the proteins or peptides.

In one aspect, a method of identifying a target protein is disclosed. In some embodiments, a target protein is identified from a complex sample mixture such as a biological sample, a blood sample, a tissue sample, a bacterial sample, or a sample of the microbiome. The presence of several, different proteins and/or peptides within a sample requires complicated techniques to isolate, purify, sequence, and identify a target peptide. The present invention discloses a method of identifying a target peptide from a plurality of peptides within a sample. In one aspect, a method of identifying a target protein comprises: (i) contacting a sample comprising the target protein with a chemical probe that comprises a functional unit that specifically interacts with the protein; (ii) loading the sample on a chip comprising a plurality of sample wells; (iii) detecting the sample on the chip, wherein a subset of the plurality of sample wells comprises the target protein bound to the chemical probe; and (iv) sequencing the contents of each of the subset of the plurality of sample wells, thereby identifying the target protein. In some embodiments, the sample is a biological sample, such as a blood sample, a tissue sample, a bacterial sample, or a sample of the microbiome. In some embodiments, the sample is a whole proteome.

In some embodiments, the target protein is contacted with a chemical probe that comprises a functional unit. The chemical probe can be a small molecule, a drug (e.g., a drug that interacts with the active site of an enzyme), a synthetic analogue of alkyl-CoAs, an NTP (e.g., ATP), or an NDP (e.g., ADP), a small molecule that binds covalently to the protein (e.g., at an active site), a small molecule that binds non-covalently to the protein (e.g., at an allosteric site). In some embodiments, the functional unit comprises a chemical warhead (e.g., electrophilic warhead, photocaged radical warhead, nucleophilic warhead). The chemical probe can further comprise an orthogonal reactive group (e.g., a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO)).

In some embodiments, the method of identifying a target protein can be automated or manual. In one embodiment, in which the method is automated, the automated method can occur in a single instrument. In one embodiment, the method of identifying a target protein requires a lower concentration of target protein compared to the concentration required by conventional LC-MS technologies.

In another aspect, a method of protein sequencing is disclosed, the method comprising: (i) immobilizing a target protein bound to a chemical probe at the base of a well within a chip comprising a plurality of wells; (ii) detecting the target protein bound to the chemical probe in a subset of the plurality of wells; and (iii) sequencing the contents of each of the subset of the plurality of wells, thereby identifying the target protein. In some embodiments, the target protein bound to a chemical probe is immobilized to the base of the well via a secondary complex. In some embodiments, the secondary complex is a streptavidin-biotin complex.

In another aspect, a chip is disclosed, the chip comprising a plurality of wells and a target protein bound to a chemical probe immobilized to the base of a subset of the plurality of wells.

In another aspect, a method of identifying two or more protein homologues is disclosed, the method comprising: (i) contacting a sample comprising the two or more protein homologues with a chemical probe that comprises a functional unit that binds to a shared feature of the two or more protein homologues; (ii) loading the sample on a chip comprising a plurality of sample wells; (iii) detecting the sample on the chip, wherein a subset of the plurality of samples wells comprises at least one of the two or more protein homologues; and (iv) sequencing the contents of each of the subset of the plurality of sample wells, thereby identifying each protein of the plurality of target proteins. In some embodiments, the two or more protein homologues comprise amino acid sequences that share at least 80%, at least 85%, at least 90%, or at least 95% sequence similarity. In some embodiments, the shared feature of the two or more protein homologues is a protein domain, active site, allosteric site, or post-translational modification.

In another aspect, a composition is disclosed, the composition comprising a peptide attached to a nucleic acid via a linker, wherein a first region of the linker is covalently attached or bound to the peptide using a functional group that specifically interacts with the peptide, and wherein a second region of the linker is covalently attached or bound to the nucleic acid using an orthogonal tag (e.g., an azide moiety). In some embodiments, the linker further comprises a secondary complex. In some embodiments, the secondary complex is a streptavidin-biotin complex. In some embodiments, the composition is immobilized on a surface (e.g., the surface of a chip well).

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### Chemical Probes

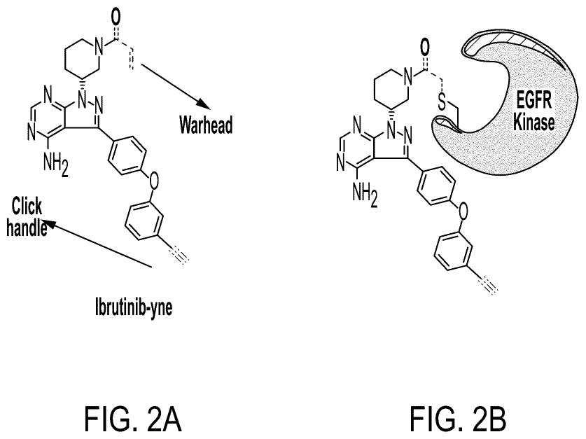

A chemical probe, as described herein, is a molecule with binding specificity for a target molecule and/or a functional unit that specifically interacts with a target molecule. In certain embodiments, the chemical probe is conjugated to the target molecule via a reaction. A chemical probe of the disclosure generally comprises a functional unit that specifically interacts with a target molecule, an orthogonal reactive group, and a linker connecting the functional unit and the orthogonal reactive group. See, for example, FIG. 2A, which shows a chemical probe (Ibrutinib-yne) comprising a functional unit that specifically interacts with a target molecule (warhead), an orthogonal reactive group (Click handle), and a linker. FIG. 2B shows a click chemistry chemical probe bound to a target molecule after a click chemistry reaction.

In certain embodiments, the reaction used to conjugate the target molecule to the chemical probe is a “click chemistry” reaction (e.g., the Huisgen alkyne-azide cycloaddition). In some embodiments, a click chemistry reaction is used to conjugate the chemical probe (e.g., after it is bound to the target molecule) to a surface (e.g., a surface of a sample well). In some embodiments, a click chemistry reaction is used to conjugate the chemical probe (e.g., after it is bound to the target molecule) to a secondary complex (e.g., a streptavidin-biotin complex). It is to be understood that any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition).

In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle,” as used herein, refers to an orthogonal reactive group that can partake in a click chemistry reaction. In some embodiments, a CLICK chemistry handle is an alkyne functional group. For example, a strained alkyne, e.g., a cyclooctyne, is a click chemistry handle, since it can partake in a strain-promoted cycloaddition. In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein. Other suitable click chemistry handles are known to those of skill in the art.

In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48: 4900-4908.

Additional click chemistry handles suitable for use in methods of conjugation described herein are well known to those of skill in the art, and such click chemistry handles include, but are not limited to, the click chemistry reaction partners, groups, and handles described in PCT/US2012/044584 and references therein, which references are incorporated herein by reference for click chemistry handles and methodology.

In some embodiments, a chemical probe comprises a functional unit that specifically interacts with a target molecule. A functional unit that specifically interacts with a target molecule may be, in some embodiments, an electrophilic warhead that can react with side chains of individual amino acids (e.g., cysteine or lysine) of a protein. In some embodiments, a functional unit that interacts with a target molecule is any chemical functional group that is capable of reacting with an N-terminus, C-terminus, backbone, or side chain of a target protein.

In some embodiments, the chemical probe comprises an alkyl group. In some embodiments, the alkyl group is a component of a linker between a functional unit that specifically interacts with a target protein and an orthogonal reactive group. In some embodiments, the alkyl group is a component of a functional unit that specifically interacts with a target protein. In some embodiments, the alkyl group is a component of an orthogonal reactive group. The term “alkyl” refers to a radical of a straight-chain or branched saturated hydrocarbon group having from 1 to 20 carbon atoms (“C1-20 alkyl”) In some embodiments, an alkyl group has 1 to 10 carbon atoms (“C1-10 alkyl”). In some embodiments, an alkyl group has 1 to 9 carbon atoms (“C1-9 alkyl”). In some embodiments, an alkyl group has 1 to 8 carbon atoms (“C1-8 alkyl”). In some embodiments, an alkyl group has 1 to 7 carbon atoms (“C1-7 alkyl”). In some embodiments, an alkyl group has 1 to 6 carbon atoms (“C1-6 alkyl”). In some embodiments, an alkyl group has 1 to 5 carbon atoms (“C1-5 alkyl”). In some embodiments, an alkyl group has 1 to 4 carbon atoms (“C1-4 alkyl”). In some embodiments, an alkyl group has 1 to 3 carbon atoms (“C1-3 alkyl”). In some embodiments, an alkyl group has 1 to 2 carbon atoms (“C1-2 alkyl”). In some embodiments, an alkyl group has 1 carbon atom (“C1 alkyl”). In some embodiments, an alkyl group has 2 to 6 carbon atoms (“C2-6 alkyl”). Examples of C1-6 alkyl groups include methyl (C1), ethyl (C2), propyl (C3) (e.g., n-propyl, isopropyl), butyl (C4) (e.g., n-butyl, tert-butyl, sec-butyl, iso-butyl), pentyl (C5) (e.g., n-pentyl, 3-pentanyl, amyl, neopentyl, 3-methyl-2-butanyl, tertiary amyl), and hexyl (C6) (e.g., n-hexyl). Additional examples of alkyl groups include n-heptyl (C7), n-octyl (C8), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C1-10 alkyl (such as unsubstituted C1-6 alkyl, e.g., —CH3 (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C1-10 alkyl (such as substituted C1-6 alkyl, e.g., —CH2F, —CHF2, —CF3 or benzyl (Bn)). An alkyl group may be branched or unbranched.

In some embodiments, the chemical probe comprises an alkenyl group. In some embodiments, the alkenyl group is a component of a linker between a functional unit that specifically interacts with a target protein and an orthogonal reactive group. In some embodiments, the alkenyl group is a component of a functional unit that specifically interacts with a target protein. In some embodiments, the alkenyl group is a component of an orthogonal reactive group. The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C1-20 alkenyl”). In some embodiments, an alkenyl group has 1 to 12 carbon atoms (“C1-12 alkenyl”). In some embodiments, an alkenyl group has 1 to 11 carbon atoms (“C1-11 alkenyl”). In some embodiments, an alkenyl group has 1 to 10 carbon atoms (“C1-10 alkenyl”). In some embodiments, an alkenyl group has 1 to 9 carbon atoms (“C1-9 alkenyl”). In some embodiments, an alkenyl group has 1 to 8 carbon atoms (“C1-8 alkenyl”). In some embodiments, an alkenyl group has 1 to 7 carbon atoms (“C1-7 alkenyl”). In some embodiments, an alkenyl group has 1 to 6 carbon atoms (“C1-6 alkenyl”). In some embodiments, an alkenyl group has 1 to 5 carbon atoms (“C1-5 alkenyl”). In some embodiments, an alkenyl group has 1 to 4 carbon atoms (“C1-4 alkenyl”). In some embodiments, an alkenyl group has 1 to 3 carbon atoms (“C1-3 alkenyl”). In some embodiments, an alkenyl group has 1 to 2 carbon atoms (“C1-2 alkenyl”). In some embodiments, an alkenyl group has 1 carbon atom (“C1 alkenyl”). The one or more carbon-carbon double bonds can be internal (such as in 2-butenyl) or terminal (such as in 1-butenyl). Examples of C1-4 alkenyl groups include methylidenyl (C1), ethenyl (C2), 1-propenyl (C3), 2-propenyl (C3), 1-butenyl (C4), 2-butenyl (C4), butadienyl (C4), and the like. Examples of C1-6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C1-20 alkenyl. In certain embodiments, the alkenyl group is a substituted C1-20 alkenyl. In an alkenyl group, a C═C double bond for which the stereochemistry is not specified (e.g., —CH═CHCH3 or) may be in the (E)- or (Z)-configuration.

In some embodiments, the chemical probe comprises an heteroalkenyl group. In some embodiments, the heteroalkenyl group is a component of a linker between a functional unit that specifically interacts with a target protein and an orthogonal reactive group. In some embodiments, the heteroalkenyl group is a component of a functional unit that specifically interacts with a target protein. In some embodiments, the heteroalkenyl group is a component of an orthogonal reactive group. The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC1-20 alkenyl”). In certain embodiments, a heteroalkenyl group refers to a group having from 1 to 12 carbon atoms, at least one double bond, and 1 or more heteroatoms within the parent chain (“heteroC1-12 alkenyl”). In certain embodiments, a heteroalkenyl group refers to a group having from 1 to 11 carbon atoms, at least one double bond, and 1 or more heteroatoms within the parent chain (“heteroC1-11 alkenyl”). In certain embodiments, a heteroalkenyl group refers to a group having from 1 to 10 carbon atoms, at least one double bond, and 1 or more heteroatoms within the parent chain (“heteroC1-10 alkenyl”). In some embodiments, a heteroalkenyl group has 1 to 9 carbon atoms at least one double bond, and 1 or more heteroatoms within the parent chain (“heteroC1-9 alkenyl”). In some embodiments, a heteroalkenyl group has 1 to 8 carbon atoms, at least one double bond, and 1 or more heteroatoms within the parent chain (“heteroC1-8 alkenyl”). In some embodiments, a heteroalkenyl group has 1 to 7 carbon atoms, at least one double bond, and 1 or more heteroatoms within the parent chain (“heteroC1-7 alkenyl”). In some embodiments, a heteroalkenyl group has 1 to 6 carbon atoms, at least one double bond, and 1 or more heteroatoms within the parent chain (“heteroC1-6 alkenyl”). In some embodiments, a heteroalkenyl group has 1 to 5 carbon atoms, at least one double bond, and 1 or 2 heteroatoms within the parent chain (“heteroC1-5 alkenyl”). In some embodiments, a heteroalkenyl group has 1 to 4 carbon atoms, at least one double bond, and 1 or 2 heteroatoms within the parent chain (“heteroC1-4 alkenyl”). In some embodiments, a heteroalkenyl group has 1 to 3 carbon atoms, at least one double bond, and 1 heteroatom within the parent chain (“heteroC1-3 alkenyl”). In some embodiments, a heteroalkenyl group has 1 to 2 carbon atoms, at least one double bond, and 1 heteroatom within the parent chain (“heteroC1-2 alkenyl”). In some embodiments, a heteroalkenyl group has 1 to 6 carbon atoms, at least one double bond, and 1 or 2 heteroatoms within the parent chain (“heteroC1-6 alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC1-20 alkenyl. In certain embodiments, the heteroalkenyl group is a substituted heteroC1-20 alkenyl.

In some embodiments, the chemical probe comprises an alkynyl group. In some embodiments, the alkynyl group is a component of a linker between a functional unit that specifically interacts with a target protein and an orthogonal reactive group. In some embodiments, the alkynyl group is a component of a functional unit that specifically interacts with a target protein. In some embodiments, the alkynyl group is a component of an orthogonal reactive group. The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C1-20 alkynyl”). In some embodiments, an alkynyl group has 1 to 10 carbon atoms (“C1-10 alkynyl”). In some embodiments, an alkynyl group has 1 to 9 carbon atoms (“C1-9 alkynyl”). In some embodiments, an alkynyl group has 1 to 8 carbon atoms (“C1-8 alkynyl”). In some embodiments, an alkynyl group has 1 to 7 carbon atoms (“C1-7 alkynyl”). In some embodiments, an alkynyl group has 1 to 6 carbon atoms (“C1-6 alkynyl”). In some embodiments, an alkynyl group has 1 to 5 carbon atoms (“C1-5 alkynyl”). In some embodiments, an alkynyl group has 1 to 4 carbon atoms (“C1-4 alkynyl”). In some embodiments, an alkynyl group has 1 to 3 carbon atoms (“C1-3 alkynyl”). In some embodiments, an alkynyl group has 1 to 2 carbon atoms (“C1-2 alkynyl”). In some embodiments, an alkynyl group has 1 carbon atom (“C1 alkynyl”). The one or more carbon-carbon triple bonds can be internal (such as in 2-butynyl) or terminal (such as in 1-butynyl). Examples of C1-4 alkynyl groups include, without limitation, methylidynyl (C1), ethynyl (C2), 1-propynyl (C3), 2-propynyl (C3), 1-butynyl (C4), 2-butynyl (C4), and the like. Examples of C1-6 alkenyl groups include the aforementioned C2-4 alkynyl groups as well as pentynyl (C5), hexynyl (C6), and the like. Additional examples of alkynyl include heptynyl (C7), octynyl (C8), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C1-20 alkynyl. In certain embodiments, the alkynyl group is a substituted C1-20 alkynyl.

In some embodiments, the chemical probe comprises an heteroalkynyl group. In some embodiments, the heteroalkynyl group is a component of a linker between a functional unit that specifically interacts with a target protein and an orthogonal reactive group. In some embodiments, the heteroalkynyl group is a component of a functional unit that specifically interacts with a target protein. In some embodiments, the heteroalkynyl group is a component of an orthogonal reactive group. The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC1-20 alkynyl”). In certain embodiments, a heteroalkynyl group refers to a group having from 1 to 10 carbon atoms, at least one triple bond, and 1 or more heteroatoms within the parent chain (“heteroC1-10 alkynyl”). In some embodiments, a heteroalkynyl group has 1 to 9 carbon atoms, at least one triple bond, and 1 or more heteroatoms within the parent chain (“heteroC1-9 alkynyl”). In some embodiments, a heteroalkynyl group has 1 to 8 carbon atoms, at least one triple bond, and 1 or more heteroatoms within the parent chain (“heteroC1-8 alkynyl”). In some embodiments, a heteroalkynyl group has 1 to 7 carbon atoms, at least one triple bond, and 1 or more heteroatoms within the parent chain (“heteroC1-7 alkynyl”). In some embodiments, a heteroalkynyl group has 1 to 6 carbon atoms, at least one triple bond, and 1 or more heteroatoms within the parent chain (“heteroC1-6 alkynyl”). In some embodiments, a heteroalkynyl group has 1 to 5 carbon atoms, at least one triple bond, and 1 or 2 heteroatoms within the parent chain (“heteroC1-5 alkynyl”). In some embodiments, a heteroalkynyl group has 1 to 4 carbon atoms, at least one triple bond, and 1 or 2 heteroatoms within the parent chain (“heteroC1-4 alkynyl”). In some embodiments, a heteroalkynyl group has 1 to 3 carbon atoms, at least one triple bond, and 1 heteroatom within the parent chain (“heteroC1-3 alkynyl”). In some embodiments, a heteroalkynyl group has 1 to 2 carbon atoms, at least one triple bond, and 1 heteroatom within the parent chain (“heteroC1-2 alkynyl”). In some embodiments, a heteroalkynyl group has 1 to 6 carbon atoms, at least one triple bond, and 1 or 2 heteroatoms within the parent chain (“heteroC1-6 alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC1-20 alkynyl. In certain embodiments, the heteroalkynyl group is a substituted heteroC1-20 alkynyl.

In some embodiments, the chemical probe comprises an alkoxy group. In some embodiments, the alkoxy group is a component of a linker between a functional unit that specifically interacts with a target protein and an orthogonal reactive group. In some embodiments, the alkoxy group is a component of a functional unit that specifically interacts with a target protein. In some embodiments, the alkoxy group is a component of an orthogonal reactive group. As used herein, the term “alkoxy” refers to an alkyl group having an oxygen atom that connects the alkyl group to the point of attachment: i.e., alkyl-O—. As for the alkyl portions, alkoxy groups can have any suitable number of carbon atoms, such as C1-6 or C1-4. Alkoxy groups include, for example, methoxy, ethoxy, propoxy, iso propoxy, butoxy, 2 butoxy, iso butoxy, sec butoxy, tert butoxy, pentoxy, hexoxy, etc. Alkoxy groups are unsubstituted, but can be described, in some embodiments as substituted. “Substituted alkoxy” groups can be substituted with one or more moieties selected from halo, hydroxy, amino, alkylamino, nitro, cyano, and alkoxy.

In some embodiments, the chemical probe comprises a cycloalkyl group. In some embodiments, the cycloalkyl group is a component of a linker between a functional unit that specifically interacts with a target protein and an orthogonal reactive group. In some embodiments, the cycloalkyl group is a component of a functional unit that specifically interacts with a target protein. In some embodiments, the cycloalkyl group is a component of an orthogonal reactive group. The term “cycloalkyl” refers to cyclic alkyl radical having from 3 to 10 ring carbon atoms (“C3-10 cycloalkyl”). In some embodiments, a cycloalkyl group has 3 to 8 ring carbon atoms (“C3-8 cycloalkyl”). In some embodiments, a cycloalkyl group has 3 to 6 ring carbon atoms (“C3-6 cycloalkyl”). In some embodiments, a cycloalkyl group has 5 to 6 ring carbon atoms (“C5-6 cycloalkyl”). In some embodiments, a cycloalkyl group has 5 to 10 ring carbon atoms (“C5-10 cycloalkyl”). Examples of C5-6 cycloalkyl groups include cyclopentyl (C5) and cyclohexyl (C5). Examples of C3-6 cycloalkyl groups include the aforementioned C5-6 cycloalkyl groups as well as cyclopropyl (C3) and cyclobutyl (C4). Examples of C3-8 cycloalkyl groups include the aforementioned C3-6 cycloalkyl groups as well as cycloheptyl (C7) and cyclooctyl (C8). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C3-10 cycloalkyl. In certain embodiments, the cycloalkyl group is substituted C3-10 cycloalkyl.

In some embodiments, the chemical probe comprises an heteroalkyl group. In some embodiments, the heteroalkyl group is a component of a linker between a functional unit that specifically interacts with a target protein and an orthogonal reactive group. In some embodiments, the heteroalkyl group is a component of a functional unit that specifically interacts with a target protein. In some embodiments, the heteroalkyl group is a component of an orthogonal reactive group. The term “heteroalkyl,” as used herein, refers to an alkyl group, as defined herein, in which one or more of the constituent carbon atoms have been replaced by a heteroatom or optionally substituted heteroatom, e.g., nitrogen, oxygen or sulfur. Heteroalkyl groups may be optionally substituted with one, two, three, or, in the case of alkyl groups of two carbons or more, four, five, or six substituents independently selected from any of the substituents described herein. Heteroalkyl group substituents include: (1) carbonyl; (2) halo; (3) C6C10 aryl; and (4) C3-C10 carbocyclyl. A heteroalkylene is a divalent heteroalkyl group.

In some embodiments, the chemical probe comprises an aryl group. In some embodiments, the aryl group is a component of a linker between a functional unit that specifically interacts with a target protein and an orthogonal reactive group. In some embodiments, the aryl group is a component of a functional unit that specifically interacts with a target protein. In some embodiments, the aryl group is a component of an orthogonal reactive group. The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-14 aryl”). In some embodiments, an aryl group has 6 ring carbon atoms (“C6 aryl”; e.g., phenyl). In some embodiments, an aryl group has 10 ring carbon atoms (“C10 aryl”; e.g., naphthyl such as 1-naphthyl and 2-naphthyl). In some embodiments, an aryl group has 14 ring carbon atoms (“C14 aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents (e.g., —F, —OH or —O(C1-6 alkyl). In certain embodiments, the aryl group is an unsubstituted C6-14 aryl. In certain embodiments, the aryl group is a substituted C6-14 aryl.

In some embodiments, the chemical probe comprises an heteroaryl group. In some embodiments, the heteroaryl group is a component of a linker between a functional unit that specifically interacts with a target protein and an orthogonal reactive group. In some embodiments, the heteroaryl group is a component of a functional unit that specifically interacts with a target protein. In some embodiments, the heteroaryl group is a component of an orthogonal reactive group. The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

In some embodiments, the chemical probe comprises an heterocyclyl group. In some embodiments, the heterocyclyl group is a component of a linker between a functional unit that specifically interacts with a target protein and an orthogonal reactive group. In some embodiments, the heterocyclyl group is a component of a functional unit that specifically interacts with a target protein. In some embodiments, the heterocyclyl group is a component of an orthogonal reactive group. The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

In some embodiments, the chemical probe comprises an amino group. In some embodiments, the amino group is a component of a linker between a functional unit that specifically interacts with a target protein and an orthogonal reactive group. In some embodiments, the amino group is a component of a functional unit that specifically interacts with a target protein. In some embodiments, the amino group is a component of an orthogonal reactive group. The term “amino,” as used herein, represents —N(RN)2, wherein each RN is, independently, H, OH, NO2, N(RN0)2, SO2ORN0, SO2RN0, SORN0, an N protecting group, alkyl, alkoxy, aryl, cycloalkyl, acyl (e.g., acetyl, trifluoroacetyl, or others described herein), wherein each of these recited RN groups can be optionally substituted; or two RN combine to form an alkylene or heteroalkylene, and wherein each RN0 is, independently, H, alkyl, or aryl. The amino groups of the disclosure can be an unsubstituted amino (i.e., —NH2) or a substituted amino (i.e., —N(RN)2).

The terms “salt thereof” or “salts thereof” as used herein refer to salts which are well known in the art. For example, Berge et al., describe pharmaceutically acceptable salts in detail in J. Pharmaceutical Sciences, 1977, 66, 1-19, incorporated herein by reference. Additional information on suitable salts can be found in Remington's Pharmaceutical Sciences, 17th ed., Mack Publishing Company, Easton, Pa., 1985, which is incorporated herein by reference. Salts of the compounds of this invention include those derived from suitable inorganic and organic acids and bases. Examples of acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

### Protein Capping

Following a reaction in which a chemical probe binds to a target molecule (e.g., via a reaction between a warhead of a chemical probe and a side chain of an amino acid of a target protein), the amino acid side chains of the target molecule with the peptide sample may be capped. In some, but not necessarily all embodiments, a peptide sample comprises a mixture comprising: a protein, a reducing agent, an amino acid side chain capping agent, and/or a protein digestion agent. In some embodiments, a peptide sample comprises a mixture comprising: a protein, a reducing agent, an amino acid side chain capping agent, and a protein digestion agent.

Any suitable amino acid side chain capping agent may be used to cap amino acid side chains of a protein within a peptide sample. In some embodiments, the amino acid side chain capping agent prevents the formation of disulfide bonds. In some embodiments, the amino acid side chain capping agent prevents the amino acid side chain from undergoing further reactivity such as nucleophile/electrophile or redox reactivity. In some embodiments, the amino acid side chain capping agent is a cysteine capping agent. In some embodiments, the amino acid side chain capping agent is a sulfhydryl-reactive alkylating reagent (e.g. a cysteine alkylation agent). For instance, in some embodiments, the amino acid side chain capping agent comprises a haloacetamide (e.g. chloroacetamide, iodoacetamide) or a haloacetate/haloacetic acid (e.g., chloroacetate/chloroacetic acid, iodoacetate/iodoacetic acid). In some embodiments, the amino acid side chain capping agent is an aromatic benzyl halide. For example, the amino acid side chain capping agent may be an aromatic benzyl halide derivative based on a benzene aromatic group, a pyridine aromatic group, a pyrazine aromatic group, and the like. Other examples of suitable cysteine alkylating agents include 4-vinylpyridine, acrylamide, and methanethiosulfonate. In some embodiments, the amino acid side chain capping agent comprises iodoacetamide. Similarly, a target molecule undergoing capping also undergoes proteolytic digestion.

### Proteolytic Digestion and Preparation

Target molecules are exposed to digestion reagents and undergo proteolytic digestion in preparation for sequencing and detection. Any suitable protein digestion method may be used, and several are described in detail below. In some specific embodiments, a protein digestion reagent is an enzymatic protein digestion reagent. For example, in some embodiments, the protein digestion agent comprises a protease. In some embodiments, the protease comprises trypsin, Lys-C, Asp-N, ArgC, chymotrypsin, LysN and/or Glu-C. In some embodiments, the protease is trypsin. In some embodiments, the protease is Lys-C. In some embodiments, the protease is Asp-N. In some embodiments, the protease is ArgC. In some embodiments, the protease is chymotrypsin. In some embodiments, the protease is LysN. In some embodiments, the protease is Glu-C.

In some embodiments described herein, peptide samples are buffered to maintain pH within particular ranges. For instance, in some embodiments, peptide samples are buffered to maintain pH greater than or equal to 6, greater than or equal to 7, greater than or equal to 8, greater than or equal to 9, greater than or equal to 10, and/or greater at room temperature. In some embodiments, peptide samples are buffered to maintain pH less than or equal to 11, less than or equal to 10, less than or equal to 9, less than or equal to 8, less than or equal to 7, and/or less at room temperature. Combinations of these ranges are possible. For example, in some embodiments, peptide samples are buffered to maintain a pH of between 6 and 9.

In some embodiments described herein, a peptide sample may be buffered to a first pH range for a first step, and buffered to a second pH range for a second step. For example, in some embodiments, a peptide sample is buffered to a pH of 6 to 9 during incubation, and is then buffered to a pH of between 10 and 11 for a derivatization step. In some embodiments, the peptide sample is buffered to a desirable pH range for three, for four, for five, for six, for seven, for eight, for nine, and/or for ten or more steps. For example, in some embodiments, a peptide sample is buffered to a pH of 6 to 9 during incubation, and is then buffered to a pH of between 10 and 11 for a derivatization step, before being buffered to a pH of 7-8 for an immobilization complex forming step and a purification step.

Peptide samples may be buffered with any buffers suitable to the desired pH range of a peptide sample. For instance, in some embodiments it may be desirable to maintain a pH of between 6 and 9 for a peptide sample. Exemplary buffers appropriate to such pH ranges may comprise: HEPES buffer, phosphate buffers (e.g. PBS), Tris, Bis-Tris, carbonate buffers (e.g. buffers comprising: carbonates, such as sodium or potassium carbonate; and/or bicarbonates, such as sodium bicarbonate), which may be used separately or in combination to stabilize pH within a desired range. In some embodiments, a buffer appropriate to such pH ranges comprises: HEPES buffer, phosphate buffers (e.g. PBS), and/or carbonate buffers (e.g. buffers comprising: carbonates, such as sodium or potassium carbonate; and/or bicarbonates, such as sodium bicarbonate). One of ordinary skill in the art would be familiar with these and many other buffer systems, and the use of un-listed buffer systems is contemplated here.

In some embodiments, a peptide sample comprises a biological sample. In some embodiments, a peptide sample comprises a whole proteome, blood, saliva, sputum, feces, urine or buccal swab sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram negative bacterial cells. In some embodiments, a sample is a purified sample proteins that have been previously extracted. A blood sample may be a freshly drawn blood sample from a subject (e.g., a human subject) or a dried blood sample (e.g., preserved on solid media (e.g. Guthrie cards)). A blood sample may comprise whole blood, serum, plasma, red blood cells, and/or white blood cells.

In some embodiments, a peptide sample (e.g., a sample comprising cells or tissue), may be prepared, e.g., lysed (e.g., disrupted, degraded and/or otherwise digested) in a process in accordance with the instant disclosure. In some embodiments, a peptide sample to be prepared, e.g., lysed, comprises cultured cells, tissue samples from biopsies (e.g., tumor biopsies from a cancer patient, e.g., a human cancer patient), or any other clinical sample. In some embodiments, a peptide sample comprising cells or tissue is lysed using any one of known physical or chemical methodologies to release a target molecule (e.g., a target protein) from said cells or tissues. In some embodiments, a peptide sample may be lysed using an electrolytic method, an enzymatic method, a detergent-based method, and/or mechanical homogenization. In some embodiments, a peptide sample (e.g., complex tissues, gram positive or gram negative bacteria) may require multiple lysis methods performed in series. In some embodiments, if a peptide sample does not comprise cells or tissue (e.g., a peptide sample comprising purified protein), a lysis step may be omitted. In some embodiments, lysis of a peptide sample is performed to isolate target protein(s). In some embodiments, a lysis method further includes use of a mill to grind a peptide sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a peptide sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

In some embodiments, a peptide sample is prepared, e.g., lysed, in the presence of a buffer system. This buffer system may be used to make a slurry of the peptide sample, to suspend the peptide sample, and/or to stabilize the peptide sample during any known lysis methodology, including those methods described herein. In some embodiments, a peptide sample is prepared, e.g., lysed, in the presence of RIPA buffer, GCI buffer that comprises Guanidine-HCl buffer, Gly-NP40 buffer, a TRIS buffer, a HEPES buffer, or any other known buffering solution.

Many of the lysis methods described herein allow for the peptide sample to be lysed by mechanically homogenizing the peptide sample such that the cell walls of the peptide sample break down. For example, methods that cause lysis by mechanical homogenization include, but are not limited to bead-beating, heating (e.g., to high temperatures sufficient to disrupt cell walls, e.g., greater than 50° C., 60° C., 70° C., 80° C., 90° C., or 95° C.), syringe/needle/microchannel passage (to cause shearing), sonication, or maceration with a grinder. In some embodiments, any lysis methodology may be combined with any other lysis methodology. For example, any lysis methodology may be combined with heating and/or sonication and/or syringe/needle/microchannel passage to quicken the rate of lysis.

In some embodiments, peptide sample preparation comprises cell disruption (i.e., subsequent removal of unwanted cell and tissue elements following lysis). In some embodiments, cell disruption involves protein precipitation. In some embodiments, following precipitation, the lysed and disrupted peptide sample is subjected to centrifugation. In some embodiments, following centrifugation, the supernatant is discarded. Precipitation can be accomplished through multiple processes, including but not limited to those methods described in Winter, D. and H. Steen (2011). “Optimization of cell lysis and protein digestion protocols for the analysis of HeLa S3 cells by LC-MS/MS.” PROTEOMICS 11(24): 4726-4730. In some embodiments, proteins or peptides are immunoprecipitated. In some embodiments, centrifugation of precipitated proteins is followed by discarding of the supernatant and subsequent washing of the pellet fraction (e.g., washing using chloroform/methanol or trichloroacetic acid).

In some embodiments, a peptide sample (e.g., a peptide sample comprising a target protein) may be purified, e.g., following lysis, in a process in accordance with the instant disclosure. In some embodiments, a peptide sample may be purified using chromatography (e.g., affinity chromatography that selectively binds the peptide sample) or electrophoresis. In some embodiments, a peptide sample may be purified in the presence of precipitating agents. In some embodiments, after a purification step or method, a peptide sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a peptide sample may be initially purified by electrophoretic passage of a peptide sample through a porous matrix (e.g., cellulose acetate, agarose, acrylamide).

In some embodiments, the target molecule(s) is fragmented/digested prior to enrichment. In some embodiments, the target molecule is fragmented/digested after enrichment. In some embodiments, the target molecule(s) is fragmented/digested without any enrichment of the target molecule(s).

In some embodiments, an incubating step comprises maintaining the peptide sample at a temperature greater than or equal to 20° C., greater than or equal to 25° C., greater than or equal to 30° C., greater than or equal to 35° C., or greater than or equal to 37° C., or greater. In some embodiments, an incubating step comprises maintaining the peptide sample at a temperature less than or equal to 70° C., less than or equal to 50° C., less than or equal to 37° C., less than or equal to 35° C., or less than or equal to 30° C. Combinations of these ranges are possible. For example, an incubating step may comprise maintaining the peptide sample at a temperature greater than or equal to 20° C. and less than or equal to 70° C. In some embodiments, an incubating step comprises maintaining the peptide sample at a temperature within the above-mentioned ranges 30 (e.g., 37° C.) for at least 1 minute, at least 2 minutes, at least 5 minutes, at least 10 minutes, at least 15 minutes, at least 20 minutes, at least 25 minutes, at least 30 minutes, at least 45 minutes, at least 1 hour, at least 2 hours, at least 3 hours, at least 4 hours, at least 5 hours, at least 6 hours, or greater. In some embodiments, an incubating step comprises maintaining the peptide sample at a temperature within the above-mentioned ranges (e.g., 37° C.) for less than or equal to 20 hours, less than or equal to 15 hours, less than or equal to 10 hours, or less. Combinations (e.g., maintaining an above-mentioned temperature for at least one minute and less than or equal to 20 hours, at least 6 hours and less than or equal to 10 hours) are possible.

Incubation may result in the digestion of a peptide sample. In general, digestion of a peptide sample can be conducted using any known method, but typically will involve a nonenzymatic or an enzymatic method. Approaches for nonenzymatic digestion include, but are not limited to, acid hydrolysis and/or cleavage using a digestion agent such as cyanogen bromide, hydroxylamine, iodosobenzoic acid, dimethyl sulfoxide-hydrochloric acid, BNPS-skatole [2-(2-nitrophenylsulfenyl)-3-methylindole], or 2-nitro-5-thiocyanobenzoic acid. Electro-physical digestion methods may be employed as well, including electrochemical oxidation and/or digestion in conjunction with microwaves.

Enzymatic methods of digestion typically utilize digestion agents such as proteases to fragment protein into component peptides. These enzymes include trypsin (which is typically favored for the size of the peptides generated and the generation of a basic residue at the carboxyl terminus of the peptide), chymotrypsin, LysC, LysN, AspN, GluC and/or ArgC. Enzymatic fragmentation/digestion methods may be selected and adjusted for ease of use, speed, automation and/or effectiveness. In some embodiments, enzymatic methods include enzyme immobilization on solid substrates. Enzymatic methods may be performed in flow (e.g., in a microfluidic channel). In some embodiments, enzymatic methods are performed in an incubation region. Digestion methods may be performed automatedly. Alternatively, or in addition, digestion methods may be performed manually. An enzymatic digestion may utilize any number or combination of enzymes and may further comprise any of the known nonenzymatic methods.

In some embodiments, a sample comprising target molecule(s) is first denatured and reduced (e.g., using acetonitrile and TCEP). In some embodiments, target molecule(s) to be fragmented are subjected to a cysteine block. In some embodiments, target molecule(s) are fragmented using a mixture of trypsin and LysC (e.g., for 120 minutes). Enzymatic reactions may be quenched (e.g., using a quenching region of a fluidic device). Following peptide preparation, the target molecules are conjugated to a streptavidin-biotin species. A streptavidin-biotin species, as described herein is a 52.8 kDa streptavidin protein bound to a plurality of biotin molecules. In some embodiments, the streptavidin protein is bound to 1, 2, 3, or 4 biotin molecules. A streptavidin-biotin species can be conjugated to a chemical probe bound to a target molecule, thus facilitating the isolation of the target molecule from within a peptide sample.

Loading Sample onto a Chip

Following preparation of the target molecule or target peptide, the target molecule or target peptide may be sequenced to identify the target molecule or target peptide derived from the plurality of peptides. Sequencing in accordance with the instant disclosure, in some aspects, may involve immobilizing a protein (e.g., a target peptide) on a surface of a substrate (e.g., of a solid support, for example a chip, for example in a sequencing device or module). In some embodiments, a protein may be immobilized on a surface of a sample well (e.g., on a bottom surface of a sample well) on a substrate. In some embodiments, the N-terminal amino acid of the protein is immobilized (e.g., attached to the surface). In some embodiments, the C-terminal amino acid of the protein is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of proteins are attached to a plurality of sample wells (e.g., with one protein attached to a surface, for example a bottom surface, of each sample well), for example in an array of sample wells on a substrate.

In some embodiments, the plurality of samples wells are contained within a chip. The chip may comprise 5-100, 25-200, 50-400, 200-500, 400-1,000, 500-1,500, 750-1,500, 900-1,200, 1,000-2,000 or more wells. In some embodiments, a chip comprises 96 wells, 384 wells, 1,536 wells, or another number of wells suitable to sequence the target molecule derived from a plurality of peptides.

In one aspect of the present disclosure, the prepared target molecules can be fluorescently labeled and immobilized on a chip comprising a plurality of wells. Further, the chip comprising the plurality of wells can be detected for the fluorescent signal and the wells comprising fluorescent signal identified for protein sequencing.

In some embodiments, a chip is a disposable chip structure. It should be understood that the following description involving detection processes using a disposable chip structure is merely exemplary and is non-limiting, and any of a variety of other suitable instruments and chip designs for detection can be used. For example, a detection process using a chip that is not disposable is also envisioned, in accordance with certain embodiments. As another example, in some embodiments, an instrument for detection (e.g., detection module) may not even require a chip, and instead include detection components (e.g., photonic elements) such as optoelectronics, semiconductor substrates, and pixels itself rather than as part such components being part of a chip. While specific chips comprising a certain number of photonic elements (e.g., semiconductor substrates, pixels) are described and illustrated below, it should be understood that the chip (or instrument) may comprise as many or as few photonic elements as desired.

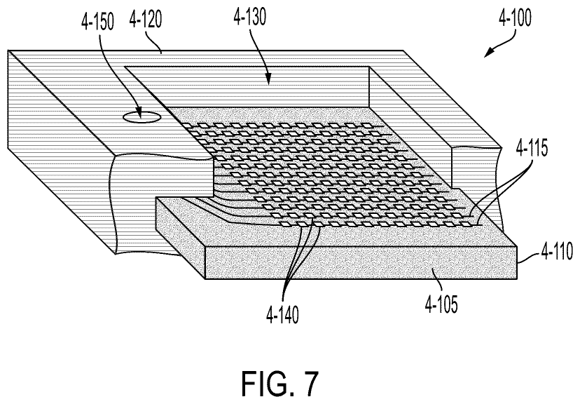

Example structure 4-100 for a disposable chip is shown in FIG. 7, according to some embodiments. The disposable chip structure 4-100 may include a bio-optoelectronic chip 4-110 having a semiconductor substrate 4-105 and including a plurality of pixels 4-140 formed on the substrate. In some embodiments, there may be row or column waveguides 4-115 that provide excitation radiation to a row or column of pixels 4-140. Excitation radiation may be coupled into the waveguides, for example, through an optical port 4-150. In some embodiments, a grating coupler may be formed on the surface of the bio-optoelectronic chip 4-110 to couple excitation radiation from a focused beam into one or more receiving waveguides that connect to the plurality of waveguides 4-115.

The disposable chip structure 4-100 may further include walls 4-120 that are formed around a pixel region on the bio-optoelectronic chip 4-110. The walls 4-120 may be part of a plastic or ceramic casing that supports the bio-optoelectronic chip 4-110. The walls 4-120 may form at least one reservoir 4-130 into which at least one sample may be placed and come into direct contact with reaction chambers on the surface of the bio-optoelectronic chip 4-110. The walls 4-120 may prevent the sample in the reservoir 4-130 from flowing into a region containing the optical port 4-150 and grating coupler, for example. In some embodiments, the disposable chip structure 4-100 may further include electrical contacts on an exterior surface of the disposable chip and interconnects within the package, so that electrical connections can be made between circuitry on the bio-optoelectronic chip 4-110 and circuitry in an instrument into which the disposable chip is mounted.

In some embodiments, a chip is as described in International Publication Number WO 2021/086985, published May 6, 2021, titled “PERISTALTIC PUMPING OF FLUIDS AND ASSOCIATED METHODS, SYSTEMS, AND DEVICES” or International Publication Number WO 2021/086990, published May 6, 2021, titled “PERISTALTIC PUMPING OF FLUIDS FOR BIOANALYTICAL APPLICATIONS AND ASSOCIATED METHODS, SYSTEMS, AND DEVICES;” the entire contents of each of which are incorporated herein by reference in their entireties.

### Peptide Sequencing and Identification

Aspects of the instant disclosure also involve methods of protein sequencing and identification, methods of amino acid identification, and compositions, systems, and devices for performing such methods. In some aspects, methods of determining the sequence of a target protein are described. In some embodiments, the target protein is enriched (e.g., enriched using electrophoretic methods, e.g., affinity SCODA) prior to determining the sequence of the target protein. In some aspects, methods of determining the sequences of a plurality of proteins (e.g., at least 2, 3, 4, 5, 10, 15, 20, 30, 50, or more) present in a sample (e.g., a purified sample, a cell lysate, a single-cell, a population of cells, or a tissue) are described. In some embodiments, a sample is prepared as described herein (e.g., digested, lysed, purified, fragmented, and/or enriched for a target protein) prior to determining the sequence of a target protein or a plurality of proteins present in a sample. In some embodiments, a target protein is an enriched target protein (e.g., enriched using electrophoretic methods, e.g., affinity SCODA). In some embodiments, the methods of protein sequencing and identification are as described in PCT/US2019/061831, filed Nov. 15, 2019, entitled “METHODS AND COMPOSITIONS FOR PROTEIN SEQUENCING,” which is incorporated herein by reference in its entirety.

In some embodiments, the instant disclosure provides methods of sequencing and/or identifying an individual protein in a sample comprising a plurality of proteins by identifying one or more types of amino acids of a protein from the mixture. In some embodiments, one or more amino acids (e.g., terminal amino acids) of the protein are labeled (e.g., directly or indirectly, for example using a binding agent) and the relative positions of the labeled amino acids in the protein are determined. In some embodiments, the relative positions of amino acids in a protein are determined using a series of amino acid labeling and cleavage steps. In some embodiments, the relative position of labeled amino acids in a protein can be determined without removing amino acids from the protein but by translocating a labeled protein through a pore (e.g., a protein channel) and detecting a signal (e.g., a Forster resonance energy transfer (FRET) signal) from the labeled amino acid(s) during translocation through the pore in order to determine the relative position of the labeled amino acids in the protein molecule.

In some embodiments, the identity of a terminal amino acid (e.g., an N-terminal or a C-terminal amino acid) is determined prior to the terminal amino acid being removed and the identity of the next amino acid at the terminal end being assessed; this process may be repeated until a plurality of successive amino acids in the protein are assessed. In some embodiments, assessing the identity of an amino acid comprises determining the type of amino acid that is present. In some embodiments, determining the type of amino acid comprises determining the actual amino acid identity (e.g., determining which of the naturally-occurring 20 amino acids an amino acid is, e.g., using a binding agent that is specific for an individual terminal amino acid). However, in some embodiments, assessing the identity of a terminal amino acid type can comprise determining a subset of potential amino acids that can be present at the terminus of the protein. In some embodiments, this can be accomplished by determining that an amino acid is not one or more specific amino acids (i.e., and therefore could be any of the other amino acids). In some embodiments, this can be accomplished by determining which of a specified subset of amino acids (e.g., based on size, charge, hydrophobicity, binding properties) could be at the terminus of the protein (e.g., using a binding agent that binds to a specified subset of two or more terminal amino acids).

In some embodiments, a protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus) as described above.

In some embodiments, a protein is sequenced from its amino (N) terminus. In some embodiments, a protein is sequenced from its carboxy (C) terminus. In some embodiments, a first terminus (e.g., N or C terminus) of a protein is immobilized and the other terminus (e.g., the C or N terminus) is sequenced as described herein.

As used herein, sequencing a protein refers to determining sequence information for a protein. In some embodiments, this can involve determining the identity of each sequential amino acid for a portion (or all) of the protein. In some embodiments, this can involve determining the identity of a fragment (e.g., a fragment of a target protein or a fragment of a sample comprising a plurality of proteins). In some embodiments, this can involve assessing the identity of a subset of amino acids within the protein and determining the relative position of one or more amino acid types without determining the identity of each amino acid in the protein). In some embodiments amino acid content information can be obtained from a protein without directly determining the relative position of different types of amino acids in the protein. The amino acid content alone may be used to infer the identity of the protein that is present (e.g., by comparing the amino acid content to a database of protein information and determining which protein(s) have the same amino acid content).

In some embodiments, sequence information for a plurality of protein fragments obtained from a target protein or sample comprising a plurality of proteins (e.g., via enzymatic and/or chemical cleavage) can be analyzed to reconstruct or infer the sequence of the target protein or plurality of proteins present in the sample. Accordingly, in some embodiments, the one or more types of amino acids are identified by detecting luminescence of one or more labeled affinity reagents that selectively bind the one or more types of amino acids. In some embodiments, the one or more types of amino acids are identified by detecting luminescence of a labeled protein.

In some embodiments, the instant disclosure provides compositions, devices, and methods for sequencing a protein by identifying a series of amino acids that are present at a terminus of a protein over time (e.g., by iterative detection and cleavage of amino acids at the terminus). In yet other embodiments, the instant disclosure provides compositions, devices, and methods for sequencing a protein by identifying labeled amino content of the protein and comparing to a reference sequence database.

In some embodiments, the instant disclosure provides compositions, devices, and methods for sequencing a protein by sequencing a plurality of fragments of the protein. In some embodiments, sequencing a protein comprises combining sequence information for a plurality of protein fragments to identify and/or determine a sequence for the protein. In some embodiments, combining sequence information may be performed by computer hardware and software. The methods described herein may allow for a set of related proteins, such as an entire proteome of an organism, to be sequenced. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells on a single chip or cartridge.

In some embodiments, methods provided herein may be used for the sequencing and identification of an individual protein in a sample comprising a plurality of proteins. In some embodiments, the instant disclosure provides methods of uniquely identifying an individual protein in a sample comprising a plurality of proteins. In some embodiments, an individual protein is detected in a mixed sample by determining a partial amino acid sequence of the protein. In some embodiments, the partial amino acid sequence of the protein is within a contiguous stretch of approximately 5-50, 10-50, 25-50, 25-100, or 50-100 amino acids.

Without wishing to be bound by any particular theory, it is expected that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a sample comprising a plurality of proteins can be fragmented (e.g., chemically degraded, enzymatically degraded) into short protein fragments of approximately 6 to 40 amino acids, and sequencing of this protein-based library would reveal the identity and abundance of each of the proteins present in the original sample. Compositions and methods for selective amino acid labeling and identifying proteins by determining partial sequence information are described in detail in U.S. patent application Ser. No. 15/510,962, filed Sep. 15, 2015, entitled “SINGLE MOLECULE PEPTIDE SEQUENCING,” which is incorporated herein by reference in its entirety.

In some embodiments, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, a protein or proteins can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, sequencing of a protein molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the protein molecule. In some embodiments, the at least two amino acids are contiguous amino acids. In some embodiments, the at least two amino acids are non-contiguous amino acids.

In some embodiments, sequencing of a protein molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the protein molecule. For example, in some embodiments, sequencing of a protein molecule comprises identification of less than 100% of one type of amino acid in the protein molecule (e.g., identification of a portion of all amino acids of one type in the protein molecule). In some embodiments, sequencing of a protein molecule comprises identification of less than 100% of each type of amino acid in the protein molecule.

In some embodiments, sequencing of a protein molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the protein.

In some embodiments, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, different characteristic patterns corresponding to different types of amino acids in a single protein may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). It should be appreciated that, in some embodiments, smaller differences in mean pulse duration between different characteristic patterns may require a greater number of pulse durations within each characteristic pattern to distinguish one from another with statistical confidence.

Sequencing of proteins in accordance with the instant disclosure, in some aspects, may be performed using a system that permits single molecule analysis. The system may include a sequencing module or device and an instrument configured to interface with the sequencing device. The sequencing module or device may include an array of pixels, where individual pixels include a sample well and at least one photodetector. The sample wells of the sequencing device may be formed on or through a surface of the sequencing device and be configured to receive a sample placed on the surface of the sequencing device. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g. a target protein). In some embodiments, the number of molecules within a sample well may be distributed among the sample wells of the sequencing device such that some sample wells contain one molecule (e.g., a target protein) while others contain zero, two, or a plurality of molecules.

In some embodiments, a sequencing module or device is positioned to receive a target molecule or sample comprising a plurality of molecules (e.g., a target protein) from a sample preparation device. In some embodiments, a sequencing device is connected directly (e.g., physically attached to) or indirectly to a sample preparation device. However, connection between the sample preparation device and the sequencing device or module (or any other type of detection module) is not necessary for all embodiments. In some embodiments, a target molecule (e.g., a target protein) or sample comprising the plurality of molecules is manually transported from the sample preparation device (e.g., sample preparation module) to the sequencing module or device either directly (e.g., without any intervening steps that change the composition of the target molecule or sample) or indirectly (e.g., involving one or more further processing steps that may change the composition of the target molecule or sample). Manual transportation may involve, for example, transport via manual pipetting or suitable manual techniques known in the art.

Excitation light is provided to the sequencing device from one or more light sources external to the sequencing device. Optical components of the sequencing device may receive the excitation light from the light source and direct the light towards the array of sample wells of the sequencing device and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a protein (or a plurality thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing module or device may include an optical system for receiving excitation light and directing the excitation light among the sample well array. The optical system may include one or more grating couplers configured to couple excitation light to the sequencing device and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides.

According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device by improving the uniformity of excitation light received by sample wells of the sequencing device. Examples of suitable components, e.g., for coupling excitation light to a sample well and/or directing emission light to a photodetector, to include in a sequencing device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing module or device may be used to position and align an excitation source to the sequencing device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing module or device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device, which may be connected to an instrument interfaced with the sequencing device. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device, such as proper alignment and/or information obtained by readout signals from the photodetectors on the sequencing device. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device, and/or data generated from the readout signals of the photodetector.

In some embodiments, the instrument may include a processing device configured to analyze data received from one or more photodetectors of the sequencing device and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target protein) or a plurality of molecules present in a sample (e.g., a plurality of proteins), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing proteins based on fluorophore decay rates and/or fluorophore intensities may facilitate a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

In some embodiments, a system comprises a detection module. The detection module may be configured to perform any of the variety of abovementioned applications (e.g., bioanalytical applications such as analysis, protein sequencing, peptide sequencing, analyte identification, diagnosis). For example, in some embodiments, the detection module comprises an analysis module. The analysis module may be configured to analyze a sample prepared by the sample preparation module. The analysis module may be configured, for example, to determine a concentration of one or more components in a fluid sample. In some embodiments, the detection module comprises a sequencing module. The sequencing module may be configured to perform sequencing of one or more components of a sample prepared by the sample preparation module. In some embodiments, the identification module is configured to identify peptide molecules (e.g., protein molecules).

A “protein,” “peptide,” or “polypeptide” (e.g., a target protein) comprises a polymer of amino acid residues linked together by peptide bonds. The terms refer to proteins, polypeptides, and peptides of any size, structure, or function. Typically, a protein or peptide will be at least three amino acids in length. In some embodiments, a peptide is between about 3 and about 100 amino acids in length (e.g., between about 5 and about 25, between about 10 and about 80, between about 15 and about 70, or between about 20 and about 40, amino acids in length). In some embodiments, a peptide is between about 6 and about 40 amino acids in length (e.g., between about 6 and about 30, between about 10 and about 30, between about 15 and about 40, or between about 20 and about 30, amino acids in length). In some embodiments, a plurality of peptides can refer to a plurality of peptide molecules, where each peptide molecule of the plurality comprises an amino acid sequence that is different from any other peptide molecule of the plurality. In some embodiments, a plurality of peptides can include at least 1 peptide and up to 1,000 peptides (e.g., at least 1 peptide and up to 10, 50, 100, 250, or 500 peptides). In some embodiments, a plurality of peptides comprises 1-5, 5-10, 1-15, 15-20, 10-100, 50-250, 100-500, 500-1,000, or more, different peptides. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein or peptide may be a fragment of a naturally occurring protein or peptide. A protein may be naturally occurring, recombinant, synthetic, or any combination of these.

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### EXAMPLES

### Example 1. Sample Preparation Workflow for the Activity-Based Protein Fingerprinting Platform

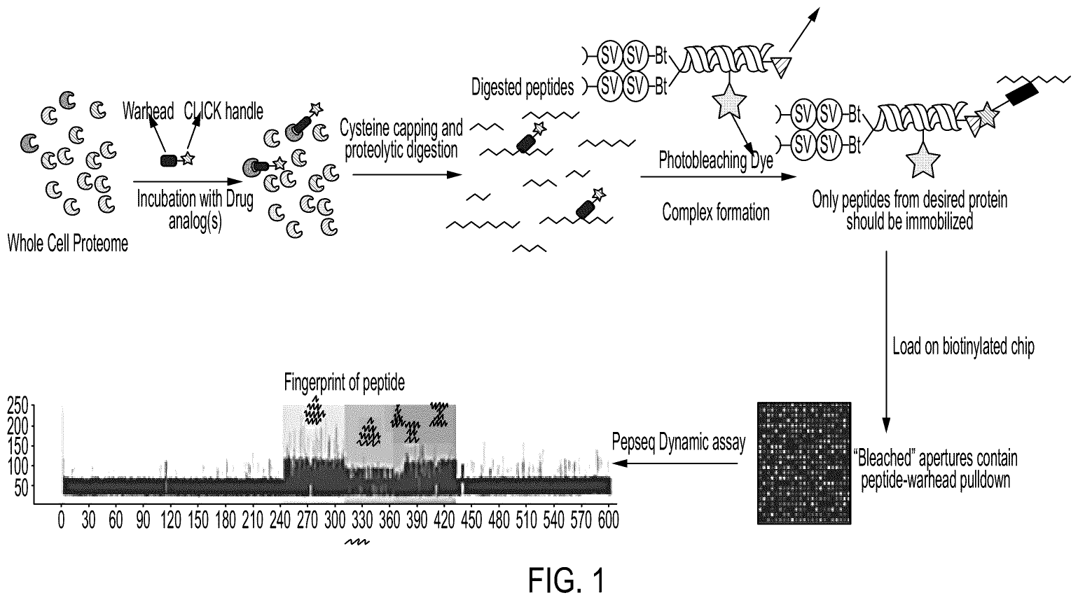

As shown in FIG. 1, a desired druggable unit comprising target peptides (e.g. whole cell proteome, blood plasma, blood serum) is exposed to a chemical probe (“drug molecule” or “drug analog”), comprising a warhead (e.g., an electrophilic warhead) and a synthetic handle capable of bioorthogonal chemistry (e.g., a CLICK handle). The druggable unit is processed by cysteine reduction, protein capping, and proteolytic digestion. Following processing, the bioorthogonal CLICK handle is used to form a peptide complex with a streptavidin immobilization complex on a chip comprising a plurality of wells.

The chip may be photobleached and each well showing a bleach output may contain the warhead covalently bound to the target peptide. Alternatively, wells containing the warhead covalently bound to the target peptide may be identified by DNA barcoding. The method of DNA barcoding that may be used as an alternative to photobleaching is further described, for example, in U.S. patent application Ser. No. 17/300,940, filed Dec. 15, 2021, titled “MOLECULAR BARCODE ANALYSIS BY SINGLE-MOLECULE KINETICS,” which is incorporated herein by reference in its entirety.

The target peptides are subsequently detected by sequencing.

### Example 2. Activity-Based Protein Fingerprinting to Detect EGFR Kinase

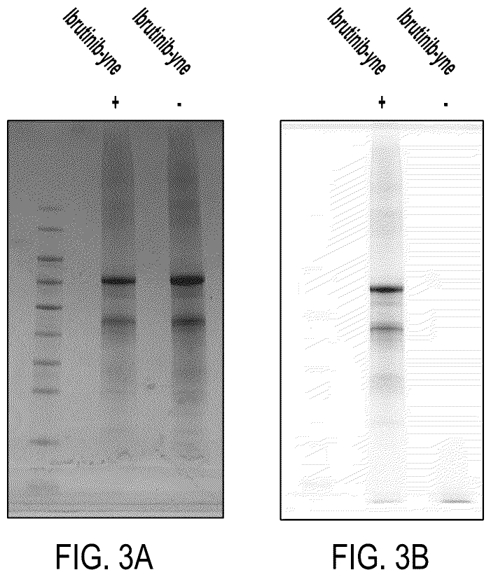

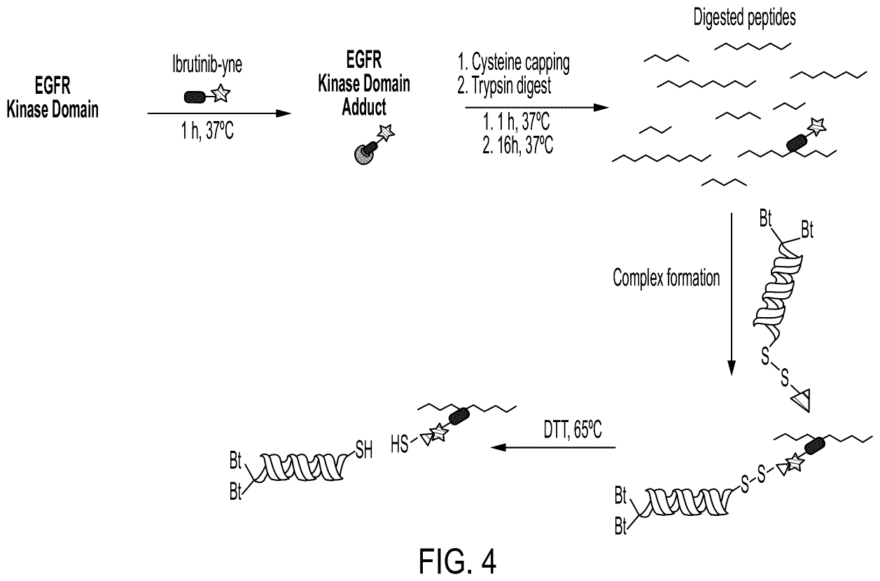

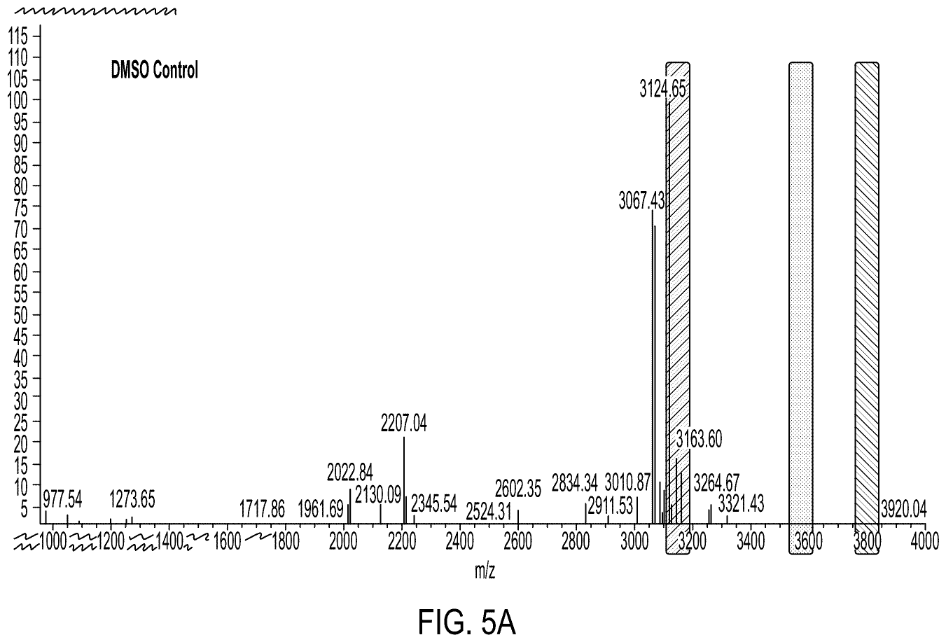

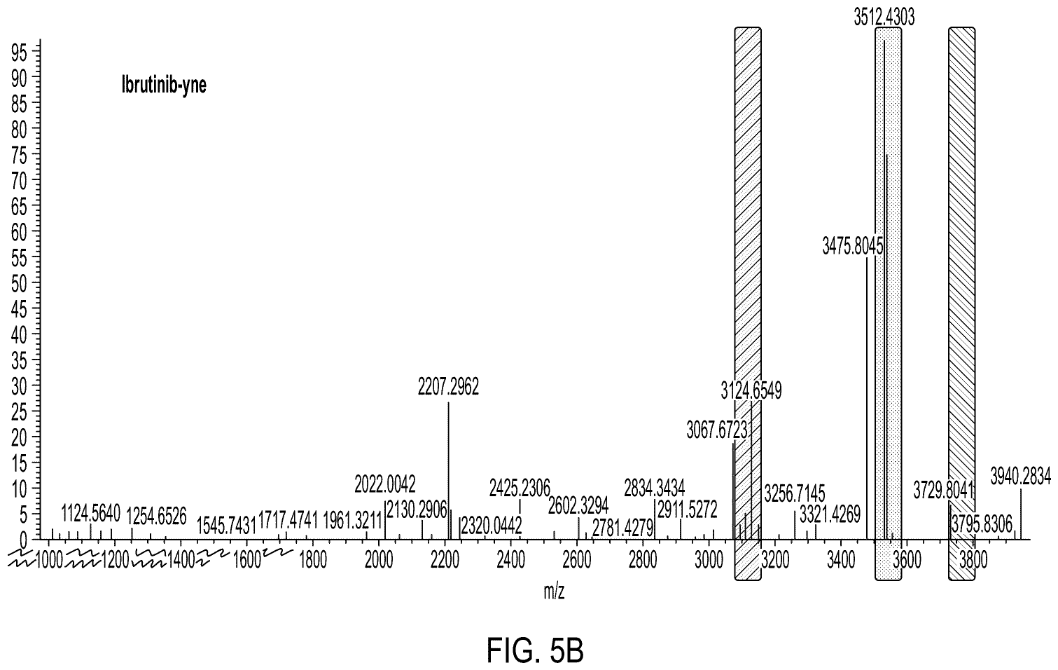

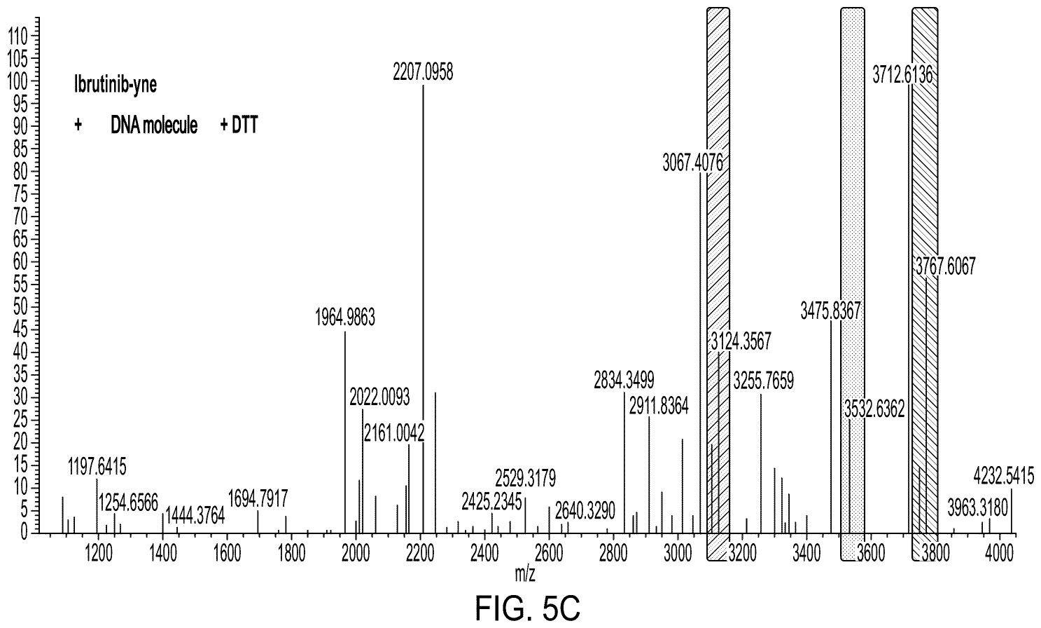

Using the steps of Example 1, EGFR kinase was detected using activity-based protein fingerprinting. The commercially available drug analog Ibrutinib-yne, which binds to the catalytic cysteine of EGFR comprises an electrophilic warhead and an alkyne moiety for the CLICK handle processing step. Purified human EGFR kinase was generated (FIG. 2A) and exposed to Ibrutinib-yne (FIG. 2B). As shown in FIG. 4, either Ibrutinib-yne or a DMSO control was incubated with EGFR at 37° C. for 1 hour. Cysteine reduction and capping of the target peptide using TCEP and chloroacetamide was conducted 37° C. for 1 hour. The complex was desalted with a 7 kDa zeba column and digested with Trypsin at 37° C. for 16 hours. Finally, the complex was conjugated with bis-biotin-DNA-SS-azide at room temperature for 30 minutes and the disulfide cleaved by incubating the complex with DTT at 65° C. for 10 minutes. The desired complex was observed by Coomassie Blue staining (FIG. 3A) and by fluorescent imaging (FIG. 3B). The target peptide detected by this protocol LLGIC(Alk)LTSTVQLITQLMPFGCLLDYVR) (SEQ ID NO:1) was detected by LC-MS (FIGS. 5A-5C). LC-MS data show that the target peptide can be detected only in its modified state in the presence of Ibrutinib-yne using this protocol.

### Example 3. Activity-Based Protein Fingerprinting to Detect a Serine Protease (Granzyme B)

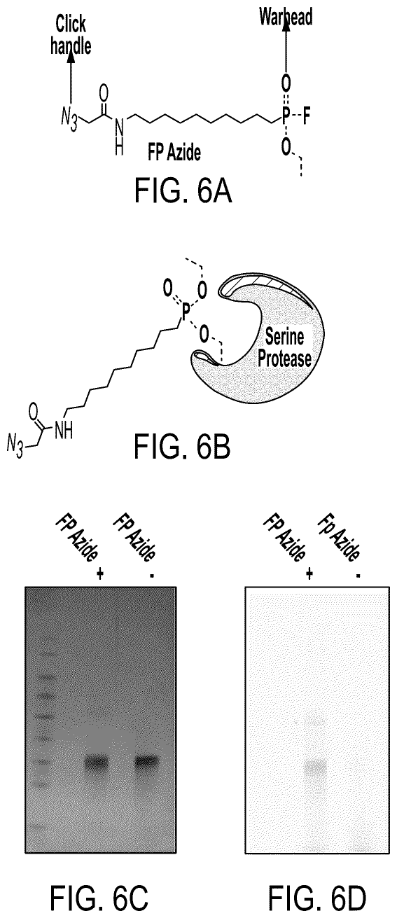

The steps of Example 1 and Example 2 were further modified to detect the serine protease Granzyme B using activity-based protein fingerprinting. A commercially available fluorophosphonate azide (FP Azide) comprising a fluorophosphonate warhead and an azide (N₃) CLICK handle (FIG. 6A) was exposed to purified Granzyme B (FIG. 6B). Either FP Azide or a DMSO control was incubated with Granzyme B at 37° C. for 1 hour. Cysteine reduction and capping of the target peptide using TCEP and chloroacetamide was conducted 37° C. for 1 hour. The complex was desalted with a 7 kDa zeba column and digested with LysC at 37° C. for 16 hours. Finally, the complex was conjugated with DNA-SS-DBCO at room temperature for 30 minutes and the disulfide cleaved by incubating the complex with DTT at 65° C. for 10 minutes. The desired complex was observed by Coomassie Blue staining (FIG. 6C) and by fluorescent imaging (FIG. 6D).

### EQUIVALENTS

Those skilled in the art will recognize or be able to ascertain using no more than routine experimentation, many equivalents to the specific embodiments of the invention described in the present application. Such equivalents are intended to be encompassed by the following claims.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

1. A method of identifying a target protein, the method comprising:
(i) contacting a sample comprising the target protein with a chemical probe that comprises a functional unit that specifically interacts with the target protein;
(ii) loading the sample on a chip comprising a plurality of sample wells;
(iii) detecting the sample on the chip, wherein at least a subset of the plurality of sample wells comprises the target protein bound to the chemical probe; and
(iv) sequencing the contents of at least the subset of the plurality of sample wells comprising the target protein bound to the chemical probe, thereby identifying the target protein.

2. The method of claim 1, wherein the sample is a biological sample or a whole proteome.

3.-4. (canceled)

5. The method of claim 1 wherein
step (i) further comprises immobilizing the target protein at a base of each of the wells of the subset of the plurality of sample wells
and step (iii) further comprises detecting the target protein.

6. The method of claim 5, wherein the target protein is immobilized to the base of the well via a secondary complex.

7.-8. (canceled)

9. The method of claim 1, wherein the chemical probe is a small molecule, wherein the small molecule is a drug, a synthetic analogue of alkyl-CoAs, a nucleoside triphosphate (NTP), a nucleoside diphosphate (NDP), a nucleoside monophosphate (NMP), a nucleotide, a small molecule that binds covalently to the target protein, at an active, or a small molecule that binds non-covalently to the target protein.

10. The method of claim 1, wherein the functional unit comprises a chemical warhead, optionally wherein the chemical warhead is an electrophilic warhead, a photocaged radical warhead, or nucleophilic warhead.

11. The method of claim 1, wherein the chemical probe further comprises an orthogonal reactive group, optionally wherein the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

12. The method of claim 1, wherein the method is automated or manual.

13. The method of claim 12, wherein the automated method occurs in a single instrument.

14. (canceled)

15. A chip comprising a plurality of wells and a target protein bound to a chemical probe immobilized to a base of at least a subset of the plurality of wells, optionally wherein the chemical probe comprises a functional unit that specifically interacts with the target protein.

16. The chip of claim 15, wherein the plurality of wells comprises at least 96 wells.

17.-18. (canceled)

19. The chip of claim 15, wherein the chemical probe is a small molecule, wherein the small molecule is a drug, a synthetic analogue of alkyl-CoAs, a nucleoside triphosphate (NTP), a nucleoside diphosphate (NDP), a nucleoside monophosphate (NMP), a nucleotide, a small molecule that binds covalently to the target protein, or a small molecule that binds non-covalently to the target protein.

20.-21. (canceled)

22. The chip of claim 15, wherein the functional unit comprises a chemical warhead, optionally wherein the chemical warhead is a an electrophilic warhead, a photocaged radical warhead, or nucleophilic warhead.

23. The chip of claim 15, wherein the chemical probe further comprises an orthogonal reactive group, optionally wherein the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

24. A method of identifying two or more protein homologues, the method comprising:
(i) contacting a sample comprising the two or more protein homologues with a chemical probe that comprises a functional unit that binds to a shared feature of the two or more protein homologues;
(ii) loading the sample on a chip comprising a plurality of sample wells;
(iii) detecting the sample on the chip, wherein at least a subset of the plurality of samples wells comprises at least one of the two or more protein homologues; and
(iv) sequencing the contents of at least the subset of the plurality of sample wells thereby identifying each protein of the plurality of target proteins.

25. The method of claim 24, wherein the two or more protein homologues comprise amino acid sequences that share at least 80%, 85%, 90%, or 95% sequence similarity.

26. The method of claim 24, wherein the shared feature of the two or more protein homologues is a protein domain, active site, allosteric site, or post-translational modification.

27. (canceled)

28. The method of claim 24, wherein the chemical probe is a small molecule, wherein the small molecule is a drug, a synthetic analogue of alkyl-CoAs, a nucleoside triphosphate (NTP), a nucleoside diphosphate (NDP), a nucleoside monophosphate (NMP), a nucleotide, a small molecule that binds covalently to the target protein, or a small molecule that binds non-covalently to the target protein.

29. The method of claim 24, wherein the functional unit comprises a chemical warhead, optionally wherein the chemical warhead is an electrophilic warhead, a photocaged radical warhead, or nucleophilic warhead.

30. The method of claim 24, wherein the chemical probe further comprises an orthogonal reactive group, optionally wherein the orthogonal reactive group is a CLICK handle, an alkyne, a cyclopropene, a tetrazine, a cyclopropene partnered with a dioxolane-fused transcyclooctene (TCO), or a tetrazine partnered with a dioxolane-fused transcyclooctene (TCO).

31.-35. (canceled)


SEQUENCE LISTING (1 sequence)

SEQ ID NO: 1 - protein, 27 aa, synthetic construct
  MOD_RES 5: note = alkyne modification
LLGICLTSTVQLITQLMPFGCLLDYVR